(19) United States
(12) Patent Application Publication
(10) Pub. No.: US 2020/0281965 A1
(43) Pub. Date: Sep. 10, 2020

(54) INHALATION OF NITRIC OXIDE

(71) Applicant: Beyond Air, Inc., Garden City, NY (US)

(72) Inventors: Amir Avniel, Ness Ziona (IL); Ali Ardakani, West Vancouver (CA); Steven A. Lisi, Rockville Centre, NY (US)

(21) Appl. No.: 16/863,935

(22) Filed: Apr. 30, 2020

Related U.S. Application Data
(63) Continuation of application No. PCT/US2018/058962, filed on Nov. 2, 2018.
(60) Provisional application No. 62/581,006, filed on Nov. 2, 2017.

(51) Int. Cl. A61K 33/00; A61K 9/00

(52) U.S. Cl. CPC A61K 33/00; A61K 9/0073

(57) ABSTRACT

A method of treating a human subject which is effected by inhalation of gaseous nitric oxide, the method comprising a first treatment period comprising administering gNO by inhalation over a period of about at least 5 days, wherein the first treatment period is followed by a second treatment period comprising administering gNO by inhalation over a period of at least 3 months. The method can be utilized for treating a human subject suffering from, or prone to suffer from, a disease or disorder that is manifested in the respiratory tract, or from a disease or disorder that can be treated via the respiratory tract. The disclosed method can be effected while monitoring one or more of on-site and off-site parameters such as vital signs, methemoglobin levels, pulmonary function parameters, blood chemistry and hematological parameters, blood coagulation parameters, inflammatory marker levels, liver and kidney function parameters and vascular endothelial activation parameters, such that no substantial deviation from a baseline in seen in one or more of the monitored parameters.

### RELATED APPLICATIONS

This application is a continuation of International Application No. PCT/US18/58962, which designated the United States and was filed on Nov. 2, 2018, published in English, which claims the benefit of U.S. Provisional Application No. 62/581,006, filed on Nov. 2, 2017. The entire teachings of the above applications are incorporated herein by reference.

### BACKGROUND OF THE INVENTION

Nitric oxide (NO) is a small lipophilic signaling molecule with a small stokes radius and a molecular weight of 30 grams/mol that enables it to cross the glycolipid cell plasma membrane into the cytosol readily and rapidly. NO has an unpaired electron available in its outer orbit that characterizes it as a free radical. NO has been shown to play a critical role in various bodily functions, including the vasodilatation of smooth muscle, neurotransmission, regulation of wound healing and immune responses to infections such as caused by bactericidal action directed toward various organisms. NO has been demonstrated to play an important role in wound healing through vasodilatation, angiogenesis, anti-inflammatory and antimicrobial action.

It has been hypothesized that the antimicrobial and cellular messenger regulatory properties of NO, delivered in an exogenous gaseous form, might easily enter the pulmonary milieu and be useful in optimizing the treatment of uncontrolled pulmonary disease with specific actions directed at reducing bacterial burden, reducing inflammation and improving clinical symptoms.

Thus there is a need for therapeutic uses of gaseous nitric oxide for treating and/or preventing various medical conditions.

### SUMMARY OF THE INVENTION

The present invention provides methods of treating and/or preventing various medical conditions, which are manifested in the respiratory tract, or which can be treated via the respiratory tract, by subjecting a human subject to gaseous nitric oxide (gNO). In particular, the present invention, in some embodiments thereof, therefore provides methods of delivering gaseous nitric oxide (gNO) to a patient the method comprising a first treatment period comprising administering gNO by inhalation over a period of about at least 5 days, wherein the first treatment period is followed by a second treatment period comprising administering gNO by inhalation over a period of at least 3 months.

### DESCRIPTION OF SPECIFIC EMBODIMENTS OF THE INVENTION

The present invention, in some embodiments thereof, relates to the delivery of gaseous nitric oxide (gNO) to a patient of delivering gaseous nitric oxide (gNO) to a patient the method comprising a first treatment period comprising administering gNO by inhalation over a period of about at least 5 days, wherein the first treatment period is followed by a second treatment period comprising administering gNO by inhalation over a period of at least 3 months. In some embodiments thereof, relates to medical treatment of respiratory diseases in human subjects, and more particularly, but not exclusively, to medical procedures based on inhalation of gaseous nitric oxide and devices for effecting the same.

Before explaining at least one embodiment of the invention in detail, it is to be understood that the invention is not limited in its application to the details set forth in the following description or exemplified by the Examples. The invention is capable of other embodiments or of being practiced or carried out in various ways.

Also, it is to be understood that the phraseology and terminology employed herein is for the purpose of description and should not be regarded as limiting, unless otherwise indicated. Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

The present invention, in some embodiments thereof, therefore provides methods of delivering gaseous nitric oxide (gNO) to a patient of delivering gaseous nitric oxide (gNO) to a patient the method comprising a first treatment period comprising administering gNO by inhalation over a period of about at least 5 days, wherein the first treatment period is followed by a second treatment period comprising administering gNO by inhalation over a period of at least 3 months.

According to embodiments of the present invention, during the first treatment period the delivery of gNO is carried out from about 5 days to about 14 days, or from 7 days to about 14 days, or from about 10 days to about 14 days. According to some embodiments of the present invention, the delivery of gNo is carried out from 7 days to 14 days. According to some embodiments of the present invention, the delivery of gNo is carried out from 10 days to 14 days. According to some embodiments of the present invention, the delivery of gNo is carried out for about 5 days. According to some embodiments of the present invention, the delivery of gNo is carried out for about 7 days. According to some embodiments of the present invention, the delivery of gNo is carried out for about 10 days. According to some embodiments of the present invention, the delivery of gNo is carried out for about 5 days.

According to embodiments of the present invention, the second treatment period immediately follows the first treatment period. In some embodiments, during the second treatment period the delivery of gNO is carried out over a period of at least 3 months. In some embodiments, during the second treatment period the delivery of gNO is carried out over a period of at least 6 months. In some embodiments, during the second treatment period the delivery of gNO is carried out over a period of at least 9 months. In some embodiments, during the second treatment period the delivery of gNO is carried out over a period of at least 12 months. In some embodiments, during the second treatment period the delivery of gNO is carried out over a period of one or more years.

In the context of embodiments of the present invention, the term “load” refers to a certain cumulative amount of nitric oxide to which a subject is exposed to during inhalation treatment (e.g., the presently claimed treatment), which is estimated in terms of ppm-hour (also referred to herein as ppm·hr), namely the average concentration of gNO in the inhalant multiplied by the overall time of exposure. The load can be estimated per cycle of the treatment (load per cycle), or per a time unit, such as a day (daily load), weekly, or total treatment period (total number of days of the treatment).

According to some embodiments of the present invention, the delivery of gNO to the subject during the first treatment period and second treatment period is independently conducted such that the subject inhales gNO at a load that ranges from about 20 ppm-hour to about 2000 ppm-hour daily. According to some embodiments of the present invention, the subject inhales gNO at a load that ranges from about 20 ppm-hour to about 1000 ppm-hour daily. According to some embodiments of the present invention, the subject inhales gNO at a load that ranges from about 20 ppm-hour to about 750 ppm-hour daily. According to some embodiments of the present invention, the subject inhales gNO at a load that ranges from about 20 ppm-hour to about 400 ppm-hour daily. According to some embodiments of the present invention, the subject inhales gNO at a load that ranges from about 20 ppm-hour to about 200 ppm-hour daily. According to some embodiments of the present invention, the subject inhales gNO at a load that ranges from about 20 ppm-hour to about 100 ppm-hour daily. According to some embodiments of the present invention, the subject inhales gNO at a load that ranges from about 20 ppm-hour to about 80 ppm-hour daily. According to some embodiments of the present invention, the subject inhales gNO at a load that ranges from about 40 ppm-hour to about 1000 ppm-hour daily. According to some embodiments of the present invention, the subject inhales gNO at a load that ranges from about 40 ppm-hour to about 750 ppm-hour daily. According to some embodiments of the present invention, the subject inhales gNO at a load that ranges from about 40 ppm-hour to about 400 ppm-hour daily. According to some embodiments of the present invention, the subject inhales gNO at a load that ranges from about 40 ppm-hour to about 200 ppm-hour daily. According to some embodiments of the present invention, the subject inhales gNO at a load that ranges from about 40 ppm-hour to about 100 ppm-hour daily. According to some embodiments of the present invention, the subject inhales gNO at a load that ranges from about 40 ppm-hour to about 80 ppm-hour daily. According to some embodiments of the present invention, the subject inhales gNO at a load that ranges from about 80 ppm-hour to about 1000 ppm-hour daily. According to some embodiments of the present invention, the subject inhales gNO at a load that ranges from about 80 ppm-hour to about 750 ppm-hour daily. According to some embodiments of the present invention, the subject inhales gNO at a load that ranges from about 80 ppm-hour to about 400 ppm-hour daily. According to some embodiments of the present invention, the subject inhales gNO at a load that ranges from about 80 ppm-hour to about 200 ppm-hour daily. According to some embodiments of the present invention, the subject inhales gNO at a load that ranges from about 80 ppm-hour to about 160 ppm-hour daily. According to some embodiments of the present invention, the subject inhales gNO at a load that ranges from about 100 ppm-hour to about 1000 ppm-hour daily. According to some embodiments of the present invention, the subject inhales gNO at a load that ranges from about 100 ppm-hour to about 750 ppm-hour daily. According to some embodiments of the present invention, the subject inhales gNO at a load that ranges from about 100 ppm-hour to about 400 ppm-hour daily. According to some embodiments of the present invention, the subject inhales gNO at a load that ranges from about 100 ppm-hour to about 200 ppm-hour daily. According to some embodiments of the present invention, the subject inhales gNO at a load of about 80 ppm-hour daily. According to some embodiments of the present invention, the subject inhales gNO at a load of about 160 ppm-hour daily. According to some embodiments of the present invention, the subject inhales gNO at a load of about 240 ppm-hour daily. According to some embodiments of the present invention, the subject inhales gNO at a load of about 320 ppm-hour daily. According to some embodiments of the present invention, the subject inhales gNO at a load of about 400 ppm-hour daily. According to some embodiments of the present invention, the subject inhales gNO at a load of about 480 ppm-hour daily. In some embodiments, the delivery is by intermittent inhalation, wherein the intermittent delivery is effected such that the daily load is inhaled in more than one session of uninterrupted administration.

In embodiments of the invention, the delivery of gNO to the patient during the first treatment period is configured to administer at least about 400 ppm·hrs to about 6,000 ppm·hrs over the treatment period. In embodiments of the invention, the delivery of gNO to the patient is configured to administer at least about 1000 ppm·hrs to about 5,800 ppm·hrs. In embodiments of the invention, the delivery of gNO to the patient is configured to administer at least about 1,500 ppm·hrs to about 5,800 ppm·hrs. In embodiments of the invention, the delivery of gNO to the patient is configured to administer at least about 2,000 ppm·hrs to about 5,800 ppm·hrs. In embodiments of the invention, the delivery of gNO to the patient is configured to administer at least about 3,200 ppm·hrs to about 5,800 ppm·hrs. In embodiments of the invention, the delivery of gNO to the patient is configured to administer at least about 1,200 ppm·hrs. In embodiments of the invention, the delivery of gNO to the patient is configured to administer at least about 1,600 ppm·hrs. In embodiments of the invention, the delivery of gNO to the patient is configured to administer at least about 2,000 ppm·hrs. In embodiments of the invention, the delivery of gNO to the patient is configured to administer at least about 2,800 ppm·hrs. In embodiments of the invention, the delivery of gNO to the patient is configured to administer at least about 3,200 ppm·hrs. In embodiments of the invention, the delivery of gNO to the patient is configured to administer at least about 4,000 ppm·hrs. In embodiments of the invention, the delivery of gNO to the patient is configured to administer at least about 5,000 ppm·hrs. In embodiments of the invention, the delivery of gNO to the patient is configured to administer at least about 5,500 ppm·hrs. In embodiments of the invention, the delivery of gNO to the patient is configured to administer at least about 5,600 ppm·hrs.

In embodiments of the invention, the delivery of gNO to the patient during the second treatment period is configured to administer at least about 7,000 ppm·hrs to about 90,000 ppm·hrs over the treatment period. In embodiments of the invention, the delivery of gNO to the patient is configured to administer at least about 14,000 ppm·hrs to about 88,000 ppm·hrs. In embodiments of the invention, the delivery of gNO to the patient is configured to administer at least about 20,000 ppm·hrs to about 87,000 ppm·hrs. In embodiments of the invention, the delivery of gNO to the patient is configured to administer at least about 7,000 ppm·hrs. In embodiments of the invention, the delivery of gNO to the patient is configured to administer at least about 7,200 ppm·hrs. In embodiments of the invention, the delivery of gNO to the patient is configured to administer at least about 14,000 ppm·hrs. In embodiments of the invention, the delivery of gNO to the patient is configured to administer at least about 14,400 ppm·hrs. In embodiments of the invention, the delivery of gNO to the patient is configured to administer at least about 20,000 ppm·hrs. In embodiments of the invention, the delivery of gNO to the patient is configured to administer at least about 21,600 ppm·hrs. In embodiments of the invention, the delivery of gNO to the patient is configured to administer at least about 28,000 ppm·hrs. In embodiments of the invention, the delivery of gNO to the patient is configured to administer at least about 28,800 ppm·hrs. In embodiments of the invention, the delivery of gNO to the patient is configured to administer at least about 40,000 ppm·hrs. In embodiments of the invention, the delivery of gNO to the patient is configured to administer at least about 43,200 ppm·hrs. In embodiments of the invention, the delivery of gNO to the patient is configured to administer at least about 55,000 ppm·hrs. In embodiments of the invention, the delivery of gNO to the patient is configured to administer at least about 57,600 ppm·hrs. In embodiments of the invention, the delivery of gNO to the patient is configured to administer at least about 60,000 ppm·hrs. In embodiments of the invention, the delivery of gNO to the patient is configured to administer at least about 64,800 ppm·hrs. In embodiments of the invention, the delivery of gNO to the patient is configured to administer at least about 72,000 ppm·hrs. In embodiments of the invention, the delivery of gNO to the patient is configured to administer at least about 80,000 ppm·hrs. In embodiments of the invention, the delivery of gNO to the patient is configured to administer at least about 86,400 ppm·hrs.

In some embodiment, the invention provides a method of delivering gaseous nitric oxide (gNO) to a patient, the method comprising administering at least 9,000 ppm·hrs of gNO by inhalation over a period of at least 3 months. In some embodiment, the invention provides a method of delivering gaseous nitric oxide (gNO) to a patient, the method comprising administering at least 10,000 ppm·hrs of gNO by inhalation over a period of at least 3 months. In some embodiment, the invention provides a method of delivering gaseous nitric oxide (gNO) to a patient, the method comprising administering at least 11,000 ppm·hrs of gNO by inhalation over a period of at least 3 months. In some embodiment, the invention provides a method of delivering gaseous nitric oxide (gNO) to a patient, the method comprising administering at least 12,000 ppm·hrs of gNO by inhalation over a period of at least 3 months. In some embodiment, the invention provides a method of delivering gaseous nitric oxide (gNO) to a patient, the method comprising administering at least 16,000 ppm·hrs of gNO by inhalation over a period of at least 3 months. In some embodiment, the invention provides a method of delivering gaseous nitric oxide (gNO) to a patient, the method comprising administering at least 17,000 ppm·hrs of gNO by inhalation over a period of at least 3 months. In some embodiment, the invention provides a method of delivering gaseous nitric oxide (gNO) to a patient, the method comprising administering at least 18,000 ppm·hrs of gNO by inhalation over a period of at least 3 months. In some embodiment, the invention provides a method of delivering gaseous nitric oxide (gNO) to a patient, the method comprising administering at least 20,000 ppm·hrs of gNO by inhalation over a period of at least 3 months. In some embodiment, the invention provides a method of delivering gaseous nitric oxide (gNO) to a patient, the method comprising administering at least 23,000 ppm·hrs of gNO by inhalation over a period of at least 3 months. In some embodiment, the invention provides a method of delivering gaseous nitric oxide (gNO) to a patient, the method comprising administering at least 24,000 ppm·hrs of gNO by inhalation over a period of at least 3 months. In some embodiment, the invention provides a method of delivering gaseous nitric oxide (gNO) to a patient, the method comprising administering at least 25,000 ppm·hrs of gNO by inhalation over a period of at least 3 months. In some embodiment, the invention provides a method of delivering gaseous nitric oxide (gNO) to a patient, the method comprising administering at least 27,000 ppm·hrs of gNO by inhalation over a period of at least 3 months.

In some embodiment, the invention provides a method of delivering gaseous nitric oxide (gNO) to a patient, the method comprising administering at least 16,000 ppm·hrs of gNO by inhalation over a period of at least 6 months. In some embodiment, the invention provides a method of delivering gaseous nitric oxide (gNO) to a patient, the method comprising administering at least 17,000 ppm·hrs of gNO by inhalation over a period of at least 6 months. In some embodiment, the invention provides a method of delivering gaseous nitric oxide (gNO) to a patient, the method comprising administering at least 18,000 ppm·hrs of gNO by inhalation over a period of at least 6 months. In some embodiment, the invention provides a method of delivering gaseous nitric oxide (gNO) to a patient, the method comprising administering at least 20,000 ppm·hrs of gNO by inhalation over a period of at least 6 months. In some embodiment, the invention provides a method of delivering gaseous nitric oxide (gNO) to a patient, the method comprising administering at least 25,000 ppm·hrs of gNO by inhalation over a period of at least 6 months. In some embodiment, the invention provides a method of delivering gaseous nitric oxide (gNO) to a patient, the method comprising administering at least 30,000 ppm·hrs of gNO by inhalation over a period of at least 6 months. In some embodiment, the invention provides a method of delivering gaseous nitric oxide (gNO) to a patient, the method comprising administering at least 31,000 ppm·hrs of gNO by inhalation over a period of at least 6 months. In some embodiment, the invention provides a method of delivering gaseous nitric oxide (gNO) to a patient, the method comprising administering at least 32,000 ppm·hrs of gNO by inhalation over a period of at least 6 months. In some embodiment, the invention provides a method of delivering gaseous nitric oxide (gNO) to a patient, the method comprising administering at least 34,000 ppm·hrs of gNO by inhalation over a period of at least 6 months. In some embodiment, the invention provides a method of delivering gaseous nitric oxide (gNO) to a patient, the method comprising administering at least 40,000 ppm·hrs of gNO by inhalation over a period of at least 6 months. In some embodiment, the invention provides a method of delivering gaseous nitric oxide (gNO) to a patient, the method comprising administering at least 45,000 ppm·hrs of gNO by inhalation over a period of at least 6 months. In some embodiment, the invention provides a method of delivering gaseous nitric oxide (gNO) to a patient, the method comprising administering at least 46,000 ppm·hrs of gNO by inhalation over a period of at least 6 months. In some embodiment, the invention provides a method of delivering gaseous nitric oxide (gNO) to a patient, the method comprising administering at least 47,000 ppm·hrs of gNO by inhalation over a period of at least 6 months. In some embodiment, the invention provides a method of delivering gaseous nitric oxide (gNO) to a patient, the method comprising administering at least 48,000 ppm·hrs of gNO by inhalation over a period of at least 6 months.

In some embodiment, the invention provides a method of delivering gaseous nitric oxide (gNO) to a patient, the method comprising administering at least 23,000 ppm·hrs of gNO by inhalation over a period of at least 9 months. In some embodiment, the invention provides a method of delivering gaseous nitric oxide (gNO) to a patient, the method comprising administering at least 24,000 ppm·hrs of gNO by inhalation over a period of at least 9 months. In some embodiment, the invention provides a method of delivering gaseous nitric oxide (gNO) to a patient, the method comprising administering at least 25,000 ppm·hrs of gNO by inhalation over a period of at least 9 months. In some embodiment, the invention provides a method of delivering gaseous nitric oxide (gNO) to a patient, the method comprising administering at least 27,000 ppm·hrs of gNO by inhalation over a period of at least 9 months. In some embodiment, the invention provides a method of delivering gaseous nitric oxide (gNO) to a patient, the method comprising administering at least 30,000 ppm·hrs of gNO by inhalation over a period of at least 9 months. In some embodiment, the invention provides a method of delivering gaseous nitric oxide (gNO) to a patient, the method comprising administering at least 35,000 ppm·hrs of gNO by inhalation over a period of at least 9 months. In some embodiment, the invention provides a method of delivering gaseous nitric oxide (gNO) to a patient, the method comprising administering at least 40,000 ppm·hrs of gNO by inhalation over a period of at least 9 months. In some embodiment, the invention provides a method of delivering gaseous nitric oxide (gNO) to a patient, the method comprising administering at least 45,000 ppm·hrs of gNO by inhalation over a period of at least 9 months. In some embodiment, the invention provides a method of delivering gaseous nitric oxide (gNO) to a patient, the method comprising administering at least 46,000 ppm·hrs of gNO by inhalation over a period of at least 9 months. In some embodiment, the invention provides a method of delivering gaseous nitric oxide (gNO) to a patient, the method comprising administering at least 47,000 ppm·hrs of gNO by inhalation over a period of at least 9 months. In some embodiment, the invention provides a method of delivering gaseous nitric oxide (gNO) to a patient, the method comprising administering at least 48,000 ppm·hrs of gNO by inhalation over a period of at least 9 months. In some embodiment, the invention provides a method of delivering gaseous nitric oxide (gNO) to a patient, the method comprising administering at least 50,000 ppm·hrs of gNO by inhalation over a period of at least 9 months. In some embodiment, the invention provides a method of delivering gaseous nitric oxide (gNO) to a patient, the method comprising administering at least 55,000 ppm·hrs of gNO by inhalation over a period of at least 9 months. In some embodiment, the invention provides a method of delivering gaseous nitric oxide (gNO) to a patient, the method comprising administering at least 60,000 ppm·hrs of gNO by inhalation over a period of at least 9 months. In some embodiment, the invention provides a method of delivering gaseous nitric oxide (gNO) to a patient, the method comprising administering at least 66,000 ppm·hrs of gNO by inhalation over a period of at least 9 months. In some embodiment, the invention provides a method of delivering gaseous nitric oxide (gNO) to a patient, the method comprising administering at least 67,000 ppm·hrs of gNO by inhalation over a period of at least 9 months. In some embodiment, the invention provides a method of delivering gaseous nitric oxide (gNO) to a patient, the method comprising administering at least 68,000 ppm·hrs of gNO by inhalation over a period of at least 9 months. In some embodiment, the invention provides a method of delivering gaseous nitric oxide (gNO) to a patient, the method comprising administering at least 70,000 ppm·hrs of gNO by inhalation over a period of at least 9 months.

In some embodiment, the invention provides a method of delivering gaseous nitric oxide (gNO) to a patient, the method comprising administering at least 30,000 ppm·hrs of gNO by inhalation over a period of at least 12 months. In some embodiment, the invention provides a method of delivering gaseous nitric oxide (gNO) to a patient, the method comprising administering at least 31,000 ppm·hrs of gNO by inhalation over a period of at least 12 months. In some embodiment, the invention provides a method of delivering gaseous nitric oxide (gNO) to a patient, the method comprising administering at least 32,000 ppm·hrs of gNO by inhalation over a period of at least 12 months. In some embodiment, the invention provides a method of delivering gaseous nitric oxide (gNO) to a patient, the method comprising administering at least 34,000 ppm·hrs of gNO by inhalation over a period of at least 12 months. In some embodiment, the invention provides a method of delivering gaseous nitric oxide (gNO) to a patient, the method comprising administering at least 40,000 ppm·hrs of gNO by inhalation over a period of at least 12 months. In some embodiment, the invention provides a method of delivering gaseous nitric oxide (gNO) to a patient, the method comprising administering at least 45,000 ppm·hrs of gNO by inhalation over a period of at least 12 months. In some embodiment, the invention provides a method of delivering gaseous nitric oxide (gNO) to a patient, the method comprising administering at least 50,000 ppm·hrs of gNO by inhalation over a period of at least 12 months. In some embodiment, the invention provides a method of delivering gaseous nitric oxide (gNO) to a patient, the method comprising administering at least 55,000 ppm·hrs of gNO by inhalation over a period of at least 12 months. In some embodiment, the invention provides a method of delivering gaseous nitric oxide (gNO) to a patient, the method comprising administering at least 59,000 ppm·hrs of gNO by inhalation over a period of at least 12 months. In some embodiment, the invention provides a method of delivering gaseous nitric oxide (gNO) to a patient, the method comprising administering at least 60,000 ppm·hrs of gNO by inhalation over a period of at least 12 months. In some embodiment, the invention provides a method of delivering gaseous nitric oxide (gNO) to a patient, the method comprising administering at least 61,000 ppm·hrs of gNO by inhalation over a period of at least 12 months. In some embodiment, the invention provides a method of delivering gaseous nitric oxide (gNO) to a patient, the method comprising administering at least 63,000 ppm·hrs of gNO by inhalation over a period of at least 12 months. In some embodiment, the invention provides a method of delivering gaseous nitric oxide (gNO) to a patient, the method comprising administering at least 65,000 ppm·hrs of gNO by inhalation over a period of at least 12 months. In some embodiment, the invention provides a method of delivering gaseous nitric oxide (gNO) to a patient, the method comprising administering at least 70,000 ppm·hrs of gNO by inhalation over a period of at least 12 months. In some embodiment, the invention provides a method of delivering gaseous nitric oxide (gNO) to a patient, the method comprising administering at least 75,000 ppm·hrs of gNO by inhalation over a period of at least 12 months. In some embodiment, the invention provides a method of delivering gaseous nitric oxide (gNO) to a patient, the method comprising administering at least 80,000 ppm·hrs of gNO by inhalation over a period of at least 12 months. In some embodiment, the invention provides a method of delivering gaseous nitric oxide (gNO) to a patient, the method comprising administering at least 85,000 ppm·hrs of gNO by inhalation over a period of at least 12 months. In some embodiment, the invention provides a method of delivering gaseous nitric oxide (gNO) to a patient, the method comprising administering at least 88,000 ppm·hrs of gNO by inhalation over a period of at least 12 months. In some embodiment, the invention provides a method of delivering gaseous nitric oxide (gNO) to a patient, the method comprising administering at least 89,000 ppm·hrs of gNO by inhalation over a period of at least 12 months. In some embodiment, the invention provides a method of delivering gaseous nitric oxide (gNO) to a patient, the method comprising administering at least 90,000 ppm·hrs of gNO by inhalation over a period of at least 12 months. In some embodiment, the invention provides a method of delivering gaseous nitric oxide (gNO) to a patient, the method comprising administering at least 92,000 ppm·hrs of gNO by inhalation over a period of at least 12 months.

According to some embodiments of the invention, delivering gNO to the patient during the first treatment period and second treatment period is independently effected by intermittently subjecting the human subject to a gaseous mixture which contains gNO at the indicated concentration (a gNO-containing gaseous mixture). The term “intermittent” as used herein means starting and ceasing an action and/or performing an action in intervals.

By “intermittent inhalation” it is meant that the subject is subjected to a gaseous mixture that contains the indicated concentration of gNO intermittently, and thus inhales such a gNO-containing gaseous mixture one or more times with intervals between each inhalation. The subject therefore inhales the gNO-containing gaseous mixture, then stops inhaling a gNO-containing gaseous mixture and inhales instead a gaseous mixture that does not contain the indicated concentration of gNO (e.g., air). In some embodiments the patient then inhales again the gNO-containing gaseous mixture followed by another inhalation of gaseous mixture that does not contain gNO, and so on and so forth.

In embodiments of the invention, the method includes administering gNO at a concentration of between about 40 ppm and about 800 ppm to the subject's lungs. In embodiments of the invention, the method includes administering gNO at a concentration of between about 40 ppm and about 400 ppm to the subject's lungs. In embodiments of the invention, the method includes administering gNO at a concentration of between about 80 ppm and about 300 ppm to the subject's lungs. In embodiments of the invention, the method includes administering gNO at a concentration of about 160 ppm to the subject's lungs. The gNO is administered by inhalation. The gNO is administered by intermittent inhalation.

The gaseous nitric oxide can be administered at a concentration between about 40 ppm and about 200 ppm. The gaseous nitric oxide can be administered at a concentration between about 80 ppm and about 200 ppm. The gaseous nitric oxide can be administered at a concentration between about 120 ppm and about 200 ppm. The gaseous nitric oxide can be administered at a concentration between about 120 ppm and about 160 ppm. The gaseous nitric oxide can be administered at a concentration between about 160 ppm and about 200 ppm. The gaseous nitric oxide can be administered at a concentration between about 200 ppm and about 300 ppm. The gaseous nitric oxide can be administered at a concentration between about 300 ppm and about 400 ppm. The gaseous nitric oxide can be administered at a concentration of 160 ppm or more. The gaseous nitric oxide can be administered at a concentration of 160 ppm.

According to some embodiments of the present invention, the delivery is effected such that the daily load is inhaled in one or more sessions of intermittent inhalation, while the load per cycle of each cycle is at least about 80 ppm-hour. Such load per cycle can be obtained, for example, by configuring the pulse(s) to deliver, during one cycle, an inhalant having 160 ppm of NO for 30 minutes (the first time period). It is noted that other concentrations and other first time periods, which afford a load of at least 80 ppm-hour per cycle, are also contemplated and encompassed by embodiments of the present invention.

In some embodiments, administration of gNO induces conformational change in the cells of the lung of the patient. In some embodiments, administration of gNO improves lung function of the patient.

In some embodiments, the patient does not have an infection.

The human subject can be subjected to the inhalation by active or passive means.

By “active means” it is meant that the gaseous mixture is administered or delivered to the respiratory tract of the human subject. This can be effected, for example, by means of an inhalation device having a delivery interface adapted for human respiratory organs.

By “passive means” it is meant that the human subject inhales a gaseous mixture containing the indicated dose of gNO without devices for delivering the gaseous mixture to the respiratory tract. For example, the subject can be subjected to gNO by entering and exiting an atmospherically controlled enclosure filled with the gNO-containing mixture of gases discussed herein, or by filling and evacuating an atmospherically controlled enclosure which is in contact with a subject's respiratory tract.

The gaseous nitric oxide can be administered at a concentration between about 40 ppm and about 200 ppm. The gaseous nitric oxide can be administered at a concentration between about 80 ppm and about 200 ppm. The gaseous nitric oxide can be administered at a concentration between about 120 ppm and about 200 ppm. The gaseous nitric oxide can be administered at a concentration between about 120 ppm and about 160 ppm. The gaseous nitric oxide can be administered at a concentration between about 160 ppm and about 200 ppm. The gaseous nitric oxide can be administered at a concentration between about 200 ppm and about 300 ppm. The gaseous nitric oxide can be administered at a concentration between about 300 ppm and about 400 ppm. The gaseous nitric oxide can be administered at a concentration of 160 ppm or more. The gaseous nitric oxide can be administered at a concentration of 160 ppm.

According to some embodiments of the present invention, the intermittent inhalation includes one or more cycles, each cycle comprising inhalation of a gaseous mixture containing gNO for a first time period, followed by inhalation of a gaseous mixture containing no gNO for a second time period. According to some embodiments of the present invention, during the second period of time the subject may inhale ambient air or a controlled mixture of gases which is devoid of gNO.

The gaseous nitric oxide can be administered for between about 1 minute and about 60 minutes. In some embodiments, the first time period spans from 10 to 45 minutes, or from 20 to 45 minutes, or from 20 to 40 minutes, and according to some embodiments, spans about 30 minutes. The gaseous nitric oxide can be administered for about 30 minutes.

According to some embodiments of the present invention, the second time period ranges from 3 to 5 hours, or from 3 to 4 hours. According to some embodiments the second time period spans about 3.5 hours.

According to some embodiments of the present invention, this inhalation regimen is repeated 1-6 times over 24 hours, depending on the duration of the first and second time periods.

In some embodiments, a cycle of intermittent delivery of gNO is repeated from 1 to 6 times a day. The cycle can be performed between 1 and 5 times per day. The cycle can be performed between 1 and 3 times per day. According to some embodiments, the cycle is repeated 5 times a day. According to some embodiments, the cycle is repeated 4 times a day. According to some embodiments, the cycle is repeated 3 times a day. According to some embodiments, the cycle is repeated 2 times a day.

In some embodiments, the methods disclosed herein are effected while monitoring various parameters relevant for maintaining the desired dosage and regimen, relevant to the safety of the procedure and relevant for efficacy of the treatment. Exemplary such parameters are those obtainable on-site in real-time, such as methemoglobin level, end-tidal CO₂ level, and oxygenation, and parameters which are obtainable off-site in the laboratory, such as blood nitrite level, urine nitrite level, and inflammatory markers' level. The present inventors have therefore demonstrated that such a method can be effected safely. Embodiments of the present invention therefore relate to methods of administering gaseous nitric oxide to human subjects in need thereof, while these parameters remain substantially unchanged.

In some embodiments, the method is carried out while maintaining a controlled mixture of inhaled and exhaled gases by standard means for monitoring and controlling, on-site, the contents and/or flow of the mixture to which the subject is subjected to, or that which is delivered through a delivery interface, and/or while monitoring on-site exhaled gases and controlling the intake by feedback in real-time. In some embodiments, the method is effected while monitoring the concentration of gNO, FiO₂/O₂, ETCO₂, and NO₂ in the gaseous mixture to which the subject is exposed or by monitoring other bodily systems non-invasively, such as blood oxygen saturation (SpO₂/SaO₂/DO) and the presence of methemoglobin in the blood (SpMet).

In some embodiments, the concentration of gNO in the gNO-containing gaseous mixture is controlled so as not to deviate from a predetermined concentration by more than 10%. For example, the method is carried out while the concentration of gNO, set to 160 ppm, does not exceed margins of 144 ppm to 176 ppm.

Similarly, in some embodiments, the NO₂ content in a gNO-containing gaseous mixture is controlled such that the concentration of NO₂ is maintained lower than 5 ppm. In some embodiments, the NO₂ content in a gNO-containing gaseous mixture is controlled such that the concentration of NO₂ is maintained lower than 3 ppm.

Further, oxygen level in the gNO-containing gaseous mixture is controlled such that the concentration of O₂ in the mixture ranges from about 20% to about 25%.

Alternatively or in addition, the oxygen level in the gNO-containing gaseous mixture is controlled such that the fraction of inspired oxygen (FiO₂) ranges from greater than about 20% to less than 100%.

The phrase “fraction of inspired oxygen” or “FiO₂”, as used herein, refers to the fraction or percentage of oxygen in a given gas sample. For example, ambient air at sea level includes 20.9% oxygen, which is equivalent to FiO₂ of 0.21. Oxygen-enriched air has a higher FiO₂ than 0.21, up to 1.00, which means 100% oxygen. In the context of embodiments of the present invention, FiO₂ is kept under 1 (less than 100% oxygen).

The phrase “end tidal CO₂” or “ETCO₂”, as used herein, refers to the partial pressure or maximal concentration of carbon dioxide (CO₂) at the end of an exhaled breath, which is expressed as a percentage of CO₂ or the pressure unit mmHg. Normal values for humans range from 5% to 6% CO₂, which is equivalent to 35-45 mmHg. Since CO₂ diffuses out of the lungs into the exhaled air, ETCO₂ values reflect cardiac output (CO) and pulmonary blood flow as the gas is transported by the venous system to the right side of the heart and then pumped to the lungs by the right ventricles. A device called capnometer measures the partial pressure or maximal concentration of CO₂ at the end of exhalation. In the context of embodiments of the present invention, ETCO₂ levels are monitored so as to afford a warning feedback when ETCO₂ is more than 60 mmHg.

Levels of respiratory NO, NO₂ and O₂ concentration levels (both inhaled and exhaled; inspiratory and expiratory gases) are typically monitored continuously by sampling from a mouthpiece sample port located in an inhalation mask NO, NO₂ and O₂ equipped with an electrochemical analyzer. In the context of embodiments of the present invention, safety considerations requires the absolute minimization of the number of occasions in which NO₂ levels exceed 5 ppm, gNO concentration variations exceeding 10%, and FiO₂/O₂ levels drop below 20% during gNO administration.

In some embodiments, the method is effected while monitoring one or more physiological parameters in the subject and while assuring that no substantial change is effected in the monitored parameters (as demonstrated herein).

In some embodiments, monitoring the one or more physiological parameters is effected by noninvasive measures and/or mild invasive measures.

In some embodiments, monitoring the physiological parameter(s) in the subject is effected by on-site measurement and analysis techniques based on samples collected sporadically, continuously or periodically from the subject on-site in real-time at the subject's bed-side, and/or off-site measurement and analysis techniques based on samples collected sporadically or periodically from the subject which are sent for processing in an off-site which provides the results and analysis at a later point in time.

In the context of some embodiments of the present invention, the phrase “on-site measurement and analysis techniques” or “on-site techniques”, refers to monitoring techniques that inform the practitioner of a given physiological parameter of the subject in real-time, without the need to send the sample or raw data to an off-site facility for analysis. On-site techniques are often noninvasive, however, some rely on sampling from an invasive medical device such as a respiratory tubus, a drainer tube, an intravenous catheter or a subcutaneous port or any other implantable probe. Thus, the phrase “on-site parameters”, as used herein, refers to physiological parameters which are obtainable by online techniques.

Other that the trivial advantage of real-time on-site determination of physiological parameters, expressed mostly in the ability of a practitioner to respond immediately and manually to any critical change thereof, the data resulting from real-time online determination of physiological parameters can be fed into the machinery and be used for real-time feedback controlling of the machinery. In the context of embodiments of the present invention, the term “real-time” also relates to systems that update information and respond thereto substantially at the same rate they receive the information. Such real-time feedback can be used to adhere to the treatment regimen and/or act immediately and automatically in response to any critical deviations from acceptable parameters as a safety measure.

Hence, according to embodiments of the present invention, the term “on-site parameter” refers to physiological and/or mechanical and/or chemical datum which is obtainable and can be put to use or consideration at or near the subject's site (e.g., bed-side) in a relatively short period of time, namely that the time period spanning the steps of sampling, testing, processing and displaying/using the datum is relatively short. An “on-site parameter” can be obtainable, for example, in less than 30 minutes, less than 10 minutes, less than 5 minutes, less than 1 minute, less than 0.5 minutes, less than 20 seconds, less than 10 seconds, less than 5 seconds, or less than 1 second from sampling to use. For example, the time period required to obtain on-site parameters by a technique known as pulse oximetry is almost instantaneous; once the device is in place and set up, data concerning, e.g., oxygen saturation in the periphery of a subject, are available in less than 1 second from sampling to use.

In the context of some embodiments of the present invention, the phrase “off-site measurement and analysis techniques” or “off-site techniques”, refers to techniques that provide information regarding a given physiological parameter of the subject after sending a sample or raw data to an offline, and typically off-site facility, and receiving the analysis offline, sometimes hours or days after the sample had been obtained. Off-site techniques are oftentimes based on samples collected by mild invasive techniques, such as blood extraction for monitoring inflammatory cytokine plasma level, and invasive techniques, such as biopsy, catheters or drainer tubus, however, some off-site techniques rely on noninvasive sampling such as urine and stool chemistry offline and off-site analyses. The phrase “off-site parameters”, as used herein, refers to physiological parameters which are obtainable by off-site laboratory techniques.

Hence, according to embodiments of the present invention, the term “off-site parameter” refers to physiological and/or mechanical and/or chemical datum which is obtain and can be put to use or consideration in a relatively long period of time, namely that the time period spanning the steps of sampling, testing, processing and displaying/using the datum is long compared to on-site parameters. Thus, an “off-site parameter” is obtainable in more than 1 day, more than 12 hours, more than 1 hour, more than 30 minutes, more than 10 minutes, or more than 5 minutes from sampling to use.

An “off-site parameter” is typically obtainable upon subjecting a sample to chemical, biological, mechanical or other procedures, which are typically performed in a laboratory and hence are not performed “on-site”, namely by or near the subject's site.

Noninvasive measures for monitoring various physiological parameters include, without limitation, pulse oximetry, nonintubated respiratory analysis and/or capnometry. Mild invasive measures for monitoring various physiological parameters include, without limitation, blood extraction, continuous blood gas and metabolite analysis, and in some embodiments intubated respiratory analysis and transcutaneous monitoring measures.

The term “pulse oximetry” refers to a noninvasive and on-site technology that measures respiration-related physiological parameters by following light absorption characteristics of hemoglobin through the skin (finger, ear lobe, etc.), and on the spectroscopic differences observed in oxygenated and deoxygenated species of hemoglobin, as well as hemoglobin species bound to other molecules, such as carbon monoxide (CO), and methemoglobin wherein the iron in the heme group is in the Fe³⁺ (ferric) state. Physiological parameters that can be determined by pulse oximetry include SpO₂, SpMet and SpCO.

The phrase “nonintubated respiratory analysis”, as used herein, refers to a group of noninvasive and on-site technologies, such as spirometry and capnography, which provide measurements of the physiological pulmonary mechanics and respiratory gaseous chemistry by sampling the inhaled/exhaled airflow or by directing subject's breath to a detector, all without entering the subject's respiratory tract or other orifices nor penetrating the skin at any stage.

The term “spirometry” as used herein, refers to the battery of measurements of respiration-related parameters and pulmonary functions by means of a noninvasive and on-site spirometer. Following are exemplary spirometry parameters which may be used in the context of some embodiments of the present invention:

The spirometric parameter Tidal volume (TV) is the amount of air inhaled and exhaled normally at rest, wherein normal values are based on person's ideal body weight.

The spirometric parameter Total Lung Capacity (TLC) is the maximum volume of air present in the lungs.

The spirometric parameter Vital Capacity (VC) is the maximum amount of air that can expel from the lungs after maximal inhalation, and is equal to the sum of inspiratory reserve volume, tidal volume, and expiratory reserve volume.

The spirometric parameter Slow Vital Capacity (SVC) is the amount of air that is inhaled as deeply as possible and then exhaled completely, which measures how deeply a person can breathe.

The spirometric parameter Forced Vital Capacity (FVC) is the volume of air measured in liters, which can forcibly be blown out after full inspiration, and constitutes the most basic maneuver in spirometry tests.

The spirometric parameter Forced Expiratory Volume in the 1st second (FEV1) is the volume of air that can forcibly be blown out in one second, after full inspiration. Average values for FEV1 in healthy people depend mainly on sex and age, whereas values falling between 80% and 120% of the average value are considered normal. Predicted normal values for FEV1 can be calculated on-site and depend on age, sex, height, weight and ethnicity as well as the research study that they are based on.

The spirometric parameter FEV1/FVC ratio (FEV1%) is the ratio of FEV1 to FVC, which in healthy adults should be approximately 75-80%. The predicted FEV1% is defined as FEV1% of the patient divided by the average FEV1% in the appropriate population for that person.

The spirometric parameter Forced Expiratory Flow (FEF) is the flow (or speed) of air coming out of the lung during the middle portion of a forced expiration. It can be given at discrete times, generally defined by what fraction remains of the forced vital capacity (FVC), namely 25% of FVC (FEF25), 50% of FVC (FEF50) or 75% of FVC (FEF75). It can also be given as a mean of the flow during an interval, also generally delimited by when specific fractions remain of FVC, usually 25-75% (FEF25-75%). Measured values ranging from 50-60% up to 130% of the average are considered normal, while predicted normal values for FEF can be calculated on-site and depend on age, sex, height, weight and ethnicity as well as the research study that they are based on. Recent research suggests that FEF25-75% or FEF25-50% may be a more sensitive parameter than FEV1 in the detection of obstructive small airway disease. However, in the absence of concomitant changes in the standard markers, discrepancies in mid-range expiratory flow may not be specific enough to be useful, and current practice guidelines recommend continuing to use FEV1, VC, and FEV1/VC as indicators of obstructive disease.

The spirometric parameter Negative Inspiratory Force (NIF) is the greatest force that the chest muscles can exert to take in a breath, wherein values indicate the state of the breathing muscles.

The spirometric parameter MMEF or MEF refers to maximal (mid-)expiratory flow and is the peak of expiratory flow as taken from the flow-volume curve and measured in liters per second. MMEF is related to peak expiratory flow (PEF), which is generally measured by a peak flow meter and given in liters per minute.

The spirometric parameter Peak Expiratory Flow (PEF) refers to the maximal flow (or speed) achieved during the maximally forced expiration initiated at full inspiration, measured in liters per minute.

The spirometric parameter diffusing capacity of carbon monoxide (DLCO) refers to the carbon monoxide uptake from a single inspiration in a standard time (usually 10 sec). On-site calculators are available to correct DLCO for hemoglobin levels, anemia, pulmonary hemorrhage and altitude and/or atmospheric pressure where the measurement was taken.

The spirometric parameter Maximum Voluntary Ventilation (MVV) is a measure of the maximum amount of air that can be inhaled and exhaled within one minute. Typically this parameter is determined over a 15 second time period before being extrapolated to a value for one minute expressed as liters/minute. Average values for males and females are 140-180 and 80-120 liters per minute respectively.

The spirometric parameter static lung compliance (Cst) refers to the change in lung volume for any given applied pressure. Static lung compliance is perhaps the most sensitive parameter for the detection of abnormal pulmonary mechanics. Cst is considered normal if it is 60% to 140% of the average value of a commensurable population.

The spirometric parameter Forced Expiratory Time (FET) measures the length of the expiration in seconds.

The spirometric parameter Slow Vital Capacity (SVC) is the maximum volume of air that can be exhaled slowly after slow maximum inhalation.

Static intrinsic positive end-expiratory pressure (static PEEPi) is measured as a plateau airway opening pressure during airway occlusion.

The spirometric parameter Maximum Inspiratory Pressure (MIP) is the value representing the highest level of negative pressure a person can generate on their own during an inhalation, which is expresented by centimeters of water pressure (cmH₂O) and measured with a manometer and serves as n indicator of diaphragm strength and an independent diagnostic parameter.

The term “capnography” refers to a technology for monitoring the concentration or partial pressure of carbon dioxide (CO₂) in the respiratory gases. End-tidal CO₂, or ETCO₂, is the parameter that can be determined by capnography.

Gas detection technology is integrated into many medical and other industrial devices and allows the quantitative determination of the chemical composition of a gaseous sample which flows or otherwise captured therein. In the context of embodiments of the present invention, such chemical determination of gases is part of the on-site, noninvasive battery of tests, controlled and monitored activity of the methods presented herein. Gas detectors, as well as gas mixers and regulators, are used to determine and control parameters such as fraction of inspired oxygen level (FiO₂) and the concentration of nitric oxide in the inhaled gas mixture.

According to some embodiments of the present invention, the measurement of vital signs, such as heart rate, blood pressure, respiratory rate and a body temperature, is regarded as part of a battery of on-site and noninvasive measurements.

The phrase “integrated pulmonary index”, or IPI, refers to a patient's pulmonary index which uses information on inhaled/exhaled gases from capnography and on gases dissolved in the blood from pulse oximetry to provide a single value that describes the patient's respiratory status. IPI, which is obtained by on-site and noninvasive techniques, integrates four major physiological parameters provided by a patient monitor (end-tidal CO₂ and respiratory rate as measured by capnography, and pulse rate and blood oxygenation SpO₂ as measured by pulse oximetry), using this information along with an algorithm to produce the IPI score. IPI provides a simple indication in real time (on-site) of the patient's overall ventilatory status as an integer (score) ranging from 1 to 10. IPI score does not replace current patient respiratory parameters, but used to assess the patient's respiratory status quickly so as to determine the need for additional clinical assessment or intervention.

According to some of any of the embodiments described herein, the monitored physiological or chemical parameters include one or more of the following parameters:

a methemoglobin level (SpMet) (an on-line parameter);

an end-tidal CO₂ level (ETCO₂) (an on-line parameter);

an oxygenation level/FIO2 or oxygen saturation level (SpO₂) (an on-line parameter);

an inflammatory cytokine plasma level (an off-line parameter); and

a serum nitrite/nitrate level (NO₂⁻/NO₃⁻) (an off-line parameter).

According to some of any of the embodiments described herein, the monitored physiological or chemical parameters further include one or more of the following parameters:

a urine level of nitrogen dioxide (urine nitrite level) (an off-line parameter);

a vital sign selected from the group consisting of a heart rate, a blood pressure, a respiratory rate and a body temperature (an on-line parameter);

a pulmonary function (spirometric parameter) (an on-line parameter) such as, but not limited to, forced expiratory volume (FEV₁), maximum mid-expiratory flow (MMEF), diffusing capacity of the lung for carbon monoxide (DLCO), forced vital capacity (FVC), total lung capacity (TLC) and residual volume (RV);

a hematological marker (an off-line parameter), such as, but not limited to, a hemoglobin level, a hematocrit ratio, a red blood cell count, a white blood cell count, a white blood cell differential and a platelet count;

a coagulation parameter (an off-line parameter) such as, but not limited to, a prothrombin time (PT), a prothrombin ratio (PR) and an international normalized ratio (INR);

a serum creatinine level (an off-line parameter);

a liver function marker (an off-line parameter) selected from the group consisting of an aspartate aminotransferase (AST) level, a serum glutamic oxaloacetic transaminase (SGOT) level, an alkaline phosphatase level, and a gamma-glutamyl transferase (GGT) level;

a vascular endothelial activation factor (an off-line parameter) selected from the group consisting of Ang-1, Ang-2 and Ang-2/Ang-1 ratio.

Non-limiting examples of inflammatory cytokines include (TNF)α, (IL)-1β, IL-6, IL-8, IL-10 and IL-12p70.

According to some embodiments of the present invention, the method as disclosed herein is such that no substantial change in at least one of the monitored parameters is observed.

In the context of the present embodiments, a change in a parameter is considered substantial when a value of an observation (measurement, test result, reading, calculated result and the likes) or a group of observations falls notably away from a normal level, for example falls about twice the upper limit of a normal level.

A “normal” level of a parameter is referred to herein as baseline values or simply “baseline”. In the context of the present embodiments, the term “baseline” is defined as a range of values which have been determined statistically from a large number of observations and/or measurements which have been collected over years of medical practice with respect to the general human population, a specific sub-set thereof (cohort) or in some cases with respect to a specific person. A baseline is a parameter-specific value which is generally and medically accepted in the art as normal for a subject under certain physical conditions. These baseline or “normal” values, and means of determining these normal values, are known in the art. Alternatively, a baseline value may be determined from or in a specific subject before effecting the method described herein using well known and accepted methods, procedures and technical means. A baseline is therefore associated with a range of tolerated values, or tolerance, which have been determined in conjunction with the measurement of a parameter. In other words, a baseline is a range of acceptable values which limit the range of observations which are considered as “normal”. The width of the baseline, or the difference between the upper and lower limits thereof are referred to as the “baseline range”, the difference from the center of the range is referred to herein as the “acceptable deviation unit” or ADU. For example, a baseline of 4-to-8 has a baseline range of 4 and an acceptable deviation unit of 2.

In the context of the present embodiments, a significant change in an observation pertaining to a given parameter is one that falls more than 2 acceptable deviation unit (2 ADU) from a predetermined acceptable baseline. For example, an observation of 10, pertaining to a baseline of 4-to-8 (characterized by a baseline range of 4, and an acceptable deviation unit of 2), falls one acceptable deviation unit, or 1 AUD from baseline. Alternatively, a change is regarded substantial when it is more than 1.5 ADU, more than 1 ADU or more than 0.5 ADU.

In the context of the present embodiments, a “statistically significant observation” or a “statistically significant deviation from a baseline” is such that it is unlikely to have occurred as a result of a random factor, error or chance.

It is noted that in some parameters or groups of parameters, the significance of a change thereof may be context-dependent, biological system-dependent, medical case-dependent, human subject-dependent, and even measuring machinery-dependent, namely a particular parameter may require or dictate stricter or looser criteria to determine if a reading thereof should be regarded as significant. It is noted herein that in specific cases some parameters may not be measurable due to patient condition, age or other reasons. In such cases the method is effected while monitoring the other parameters.

A deviation from a baseline is therefore defined as a statistically significant change in the value of the parameter as measured during and/or following a full term or a part term of administration the regimen described herein, compared to the corresponding baseline of the parameter. It is noted herein that observations of some parameters may fluctuate for several reasons, and a determination of a significant change therein should take such events into consideration and correct the appropriate baseline accordingly.

According to some embodiments of the present invention, the method comprises monitoring at least one of the parameters described hereinabove.

According to some embodiments, the monitored parameter is methemoglobin level.

As methemoglobin levels can be measured using noninvasive measures, the parameter of percent saturation at the periphery of methemoglobin (SpMet) is used to monitor the stability, safety and effectiveness of the method presented herein. Hence, according to some embodiments of the present invention, the followed parameter is SpMet and during and following the administration, the SpMet level does not exceed 5%, and preferably does not exceed 1%. As demonstrated in the Examples section that follows, a SpMet level of subjects undergoing the method described herein does not exceed 1%.

According to some embodiments, the monitored parameter is serum nitrate/nitrite level.

High nitrite and nitrate levels in a subject's serum are associated with NO toxicity and therefore serum nitrite/nitrate levels are used to detect adverse effects of the method presented herein. According to some embodiments of the present invention, the tested parameter is serum nitrite/nitrate, which is monitored during and following the treatment and the acceptable level of serum nitrite is less than 2.5 micromole/liter and serum nitrate is less than 25 micromole/liter.

According to some embodiments, the monitored parameter is level of inflammatory markers.

An elevation of inflammatory markers is associated with a phenomenon called “cytokine storm”, which has been observed in subjects undergoing gNO inhalation treatment.

Monitoring inflammatory markers while performing the method as described herein has never been taught heretofore. Moreover, methods involving gNO inhalation at a regimen in which no significant change in inflammatory markers is observed have never been taught heretofore.

According to some embodiments, the method comprises monitoring at least two of the above-mentions parameters.

In some of these embodiments, the monitored parameters are two or all of methemoglobin level, serum nitrite level and inflammatory markers.

While changes in methemoglobin level, serum nitrite level and inflammatory markers are typically observed in subjects subjected to gNO inhalation, the findings that no substantial change in these parameters has been observed in human subjects undergoing the disclosed regimen are surprising.

Hence, according to some embodiments of the present invention, the method as disclosed herein is carried out while monitoring the methemoglobin level (SpMet), the serum nitrite level (NO₂⁻) and a group of inflammatory cytokine plasma level, such as, but not limited to, (TNF)α, (IL)-1β, IL-6, IL-8, IL-10 and IL-12p70 serum levels in the subject, wherein a change in at least one of these parameters is less than 2 acceptable deviation units from a baseline.

According to some of any of the embodiments described herein, the method is effected while monitoring at least one, at least two, or all on-site parameters which include SpMet, SpO₂ and ETCO₂, and/or monitoring at least one or all off-site parameters which include serum nitrite/nitrate level and inflammatory cytokines in the plasma.

For example, the method is effected while monitoring SpMet as an on-site parameter. Alternatively, the method is effected while monitoring SpMet and ETCO₂ as on-site parameters. Alternatively, the method is effected while monitoring SpMet, ETCO₂ and SpO₂ as on-site parameters.

Further alternatively, the method is effected while monitoring SpMet as one on-site parameter, and inflammatory cytokines in the plasma as one off-site parameter. Alternatively, the method is effected while monitoring SpMet and ETCO₂ as on-site parameters, and serum nitrite/nitrate level as one off-site parameter. Alternatively, the method is effected while monitoring SpMet as one on-site parameter, and inflammatory cytokines in the plasma and serum nitrite/nitrate level as off-site parameters. Alternatively, the method is effected while monitoring ETCO₂ as one on-site parameter, and inflammatory cytokines in the plasma and serum nitrite/nitrate level as off-site parameters. Alternatively, the method is effected while monitoring SpO₂ as one on-site parameter, and inflammatory cytokines in the plasma and serum nitrite/nitrate level as off-site parameters.

Further alternatively, the method is effected while monitoring SpMet, ETCO₂ and SpO₂ as on-site parameters, and inflammatory cytokines in the plasma and serum nitrite/nitrate level as off-site parameters.

According to some of any of the embodiments described herein, the method is effected while monitoring at least one, at least two, or all on-site parameters which include SpMet, SpO₂ and ETCO₂, and/or monitoring at least one or all off-site parameters which include serum nitrite/nitrate level and inflammatory cytokines in the plasma, and further monitoring one or more and in any combination of:

a urine NO₂ level (an off-line parameter);

a vital sign (an on-line parameter);

a pulmonary function (an on-line parameter);

a hematological marker (an off-line parameter);

a coagulation parameter (an off-line parameter);

a serum creatinine level (an off-line parameter);

a liver function marker (an off-line parameter);

a vascular endothelial activation factor (an off-line parameter).

According to some of any of the embodiments described herein, the method is effected while monitoring at least one, at least two, or all on-site chemical parameters in the inhaled gas mixture, such as FiO₂ and NO₂.

It is noted herein that for any of the abovementioned embodiments, that the method is effected while no substantial change is observed in any one or more than one or all of the monitored parameters described herein.

According to some embodiments of the present invention, the method is effected while monitoring urine nitrite levels, such that the urine nitrite level is substantially unchanged during and subsequent to carrying out the method as presented herein. It is noted herein that urine nitrite levels may fluctuate for several known reasons, and a determination of a significant change therein should take such events into consideration and correct the appropriate baseline accordingly.

It is noted that urine nitrite level is indicative for the safety of gNO inhalation, yet, has never been monitored heretofore in the context of gNO inhalation in general and in the context of intermittent gNO inhalation as disclosed herein.

According to some embodiments of the present invention, hematological markers, such as the hemoglobin level, the hematocrit ratio, the red blood cell count, the white blood cell count, the white blood cell differential and the platelet count, are substantially unchanged during and subsequent to carrying out the method as presented herein.

According to some embodiments of the present invention, vascular endothelial activation factors, such as Ang-1, Ang-2 and Ang-2/Ang-1 ratio, as well as the serum creatinine level and various liver function markers, such as the aspartate aminotransferase (AST) level, the serum glutamic oxaloacetic transaminase (SGOT) level, the alkaline phosphatase level, and the gamma-glutamyl transferase (GGT) level, are substantially unchanged during and subsequent to carrying out the method as presented herein.

Oxygenation of the subject can be assessed by measuring the subject's saturation of peripheral oxygen (SpO₂). This parameter is an estimation of the oxygen saturation level, and it is typically measured using noninvasive measures, such as a pulse oximeter device. Hence, according to some embodiments of the present invention, the followed parameter during and following the administration is SpO₂, and the level of SpO₂ is higher than about 89%.

According to some embodiments of the present invention, various vital signs, such as the heart rate, the blood pressure, the respiratory rate and the body temperature; and/or various pulmonary functions (spirometric parameter), such as forced expiratory volume (FEV₁), maximum mid-expiratory flow (MMEF), diffusing capacity of the lung for carbon monoxide (DLCO), forced vital capacity (FVC), total lung capacity (TLC) and residual volume (RV); and various coagulation parameters, such as the prothrombin time (PT), the prothrombin ratio (PR) and the international normalized ratio (INR), are substantially unchanged during and subsequent to carrying out the method as presented herein. It is noted that these parameters are regarded as an indication that the general health of the subject is not deteriorating as a result of the medical condition and/or the treatment.

According to some embodiments, the aforementioned general health indicators show an improvement during and subsequent to carrying out the method as presented herein, indicating that the treatment is beneficial to the subject.

Thus, according to some embodiments of the present invention, the method as disclosed herein is effected such that general health indicators as described herein are at least remained unchanged or are improved.

According to some embodiments of the present invention, a human subject in need of gNO inhalation treatment is a human that suffers from a disease or disorder of the respiratory tract.

As used herein, the phrase “respiratory tract” encompasses all organs and tissues that are involved in the process of respiration in a human subject or other mammal subject, including cavities connected to the respiratory tract such as ears and eyes.

A respiratory tract, as used herein, encompasses the upper respiratory tract, including the nose and nasal passages, prenasal sinuses, pharynx, larynx, trachea, bronchi, and nonalveolar bronchioles; and the lower respiratory tract, including the lungs and the respiratory bronchioles, alveolar ducts, alveolar sacs, and alveoli therein.

Respiratory diseases and disorders which are treatable by any of the methods presented herein, can be classified as: Inflammatory lung disease; Obstructive lung diseases such as COPD; Restrictive lung diseases; Respiratory tract infections, such as upper/lower respiratory tract infections, and malignant/benign tumors; Pleural cavity diseases; pulmonary vascular diseases; and Neonatal diseases.

According to embodiments of the present invention, restrictive diseases include intrinsic restrictive diseases, such as asbestosis caused by long-term exposure to asbestos dust; radiation fibrosis, usually from the radiation given for cancer treatment; certain drugs such as amiodarone, bleomycin and methotrexate; as a consequence of another disease such as rheumatoid arthritis; hypersensitivity pneumonitis due to an allergic reaction to inhaled particles; acute respiratory distress syndrome (ARDS), a severe lung condition occurring in response to a critical illness or injury; infant respiratory distress syndrome due to a deficiency of surfactant in the lungs of a baby born prematurely; idiopathic pulmonary fibrosis; idiopathic interstitial pneumonia, of which there are several types; sarcoidosis; eosinophilic pneumonia; lymphangioleiomyomatosis; pulmonary Langerhans' cell histiocytosis; pulmonary alveolar proteinosis; interstitial lung diseases (ILD) such as inhaled inorganic substances: silicosis, asbestosis, berylliosis, inhaled organic substances: hypersensitivity pneumonitis, drug induced: antibiotics, chemotherapeutic drugs, antiarrhythmic agents, statins, connective tissue disease: Systemic sclerosis, polymyositis, dermatomyositis, systemic lupus erythematosus, rheumatoid arthritis, infection, atypical pneumonia, pneumocystis pneumonia (PCP), tuberculosis, Chlamydia trachomatis, RSV, idiopathic sarcoidosis, idiopathic pulmonary fibrosis, Hamman-Rich syndrome, antisynthetase syndrome, and malignant lymphangitic carcinomatosis; and extrinsic restrictive diseases, such as neuromuscular diseases, including Myasthenia gravis and Guillain barre; nonmuscular diseases of the upper thorax such as kyphosis and chest wall deformities; diseases restricting lower thoracic/abdominal volume due to obesity, diaphragmatic hernia, or the presence of ascites; and pleural thickening.

According to embodiments of the present invention, obstructive diseases include asthma, COPD, chronic bronchitis, emphysema, bronchiectasis, CF, and bronchiolitis.

Respiratory diseases and disorders which are treatable by any of the methods presented herein, can also be classified as acute or chronic; caused by an external factor or an endogenous factor; or as infectious or noninfectious respiratory diseases and disorders.

Diseases and disorders of the respiratory tract include otolaryngological and/or an upper respiratory tract and/or a lower respiratory system diseases and disorders, and are also referred to herein as “respiratory diseases” or “respiratory diseases and disorders”.

Exemplary, and most common, diseases and disorders of the respiratory tract include acute infections, such as, for example, sinusitis, broncholitis, tubercolosis, pneumonia, bronchitis, and influenza, and chronic conditions such as asthma, CF and chronic obstructive pulmonary disease.

According to some embodiments of the present invention, subject in need of gNO inhalation treatment is a human subject that suffers from a disease or disorder that is manifested in the respiratory tract, as defined herein.

In any of the embodiments described herein a human subject includes any living human at any age, from neonatals and newborns, to adults and elderly people, at any weight, height, and any other physical state.

A disease or disorder that is manifested in the respiratory tract encompasses also any disease or disorder that is not caused by an infection or airway obstruction in the respiratory tract, rather, is caused by another factor yet can be manifested by an infection or airway obstruction in the respiratory tract.

An exemplary such condition is cystic fibrosis (CF). CF is a genetic disorder in which mutations in the epithelial chloride channel, CF transmembrane conductance regulator (CFTR), impairs various mechanism of innate immunity. Chronic microbial lung infections are the leading cause of morbidity and mortality in CF patients. Early antibiotic eradication treatment of CF patients for the most prevalent bacterial pathogen, Pseudomonas aeruginosa, has considerably increased the life expectancy in CF, however still the vast majority of adult CF patients suffer from chronic P. aeruginosa lung infections which are difficult to treat due to biofilm formation and the development of antibiotic resistant strains of the virulent. Other species found in CF airways include antibiotic resistant strains such as methicillin-resistant S. aureus (MRSA), members of the Burkholderia cepacia complex, Haemophilus influenzae, Stenotrophomonas maltophilia, Achromobacter xylosoxidans, non-tuberculous mycobacteria (NTM) species and various strict anaerobic bacteria.

According to some embodiments of the present invention, a human subject in need of gNO inhalation treatment is a human subject that is prone to suffer from a respiratory tract disease or disorder. By “prone to suffer” it is meant that the human subject is at a higher risk of suffering from the disease or disorder compared to a normal subject.

Such human subjects include, for example, immuno-compromised subjects such as subjects having HIV, cancer patients undergoing or which underwent chemotherapy, cancer and other patients undergoing or which underwent transplantation, including bone marrow transplantation and transplantation of a solid organ, subjects with chronic asthma or sinusitis, and subjects which were in contact with subject(s) afflicted by an infectious respiratory tract disease or disorder, or which have otherwise been exposed to a pathogen. It is noted herein that subjecting a human subject prone to suffer from a respiratory tract disease or disorder to the gNO inhalation treatment presented herein, can be regarded as a preventative treatment, preventive care, or as a prophylactic medical treatment.

Alternatively, a human subject in need of gNO treatment is an immuno-compromised subject such as subjects having HIV, cancer patients undergoing or which underwent chemotherapy, cancer and other patients undergoing or which underwent transplantation, including bone marrow transplantation and transplantation of a solid organ, which have been infected or otherwise suffer from a respiratory disease or disorder as described herein.

Exemplary diseases or disorders of such immune-compromised subjects are described in more detail hereinbelow.

According to some embodiments of the present invention, a human subject in need of gNO inhalation treatment is a human subject that suffers from a disease or disorder that is treatable via the respiratory tract.

Since inhaled gNO is absorbed in the lungs, it contacts the blood system and hence can reach other tissues and organs in the biological system. Thus, diseases and disorders that are not associated directly to the respiratory tract, yet can be treated by inhalation of agents that show therapeutic effect on such diseases and disorders, can be treated according to embodiments of the present invention. Exemplary such diseases and disorders include, but are not limited to, acidosis, sepsis, leishmaniasis, and various viral infections.

The parasite family, Leishmania, has been extensively studied in the literature which shows that gNO kills the parasite directly. Leishmania parasites preferentially infect macrophages. Infection by Leishmania causes the macrophage to produce IFN-gamma which induces the production of iNOS, an enzyme responsible for the production of nitric oxide. However, certain presentations of Leishmania cause the macrophage to also produce IL-10 and TGF-Beta which both minimize the induction of iNOS. The decrease in NO levels is a key factor allowing the infection to continue. It would therefore be highly beneficial to determine if treatment with gNO inhalation circumvents the defense system of the parasite. Nonetheless, gNO administered by inhalation at any concentration has not been demonstrated as safe or effective against leishmaniasis hitherto.

Additional such diseases and disorders include viral infections. Viruses have been and most likely will stay a challenging “moving target” for modern medicinal methodologies. Without cell walls and thiol based detoxification pathways, viruses may be inherently more susceptible to nitrosative stress. Several in-vitro studies, using NO donors, as oppose to gNO, have demonstrated that NO inhibits viral ribonucleotide reductase, a necessary constituent enzyme of viral DNA synthesis and therefore inhibit viral replication. It has been demonstrated that NO inhibits the replication of viruses early during the replication cycle, involving the synthesis of vRNA and mRNA encoding viral proteins. Other direct mechanisms could also account for the viricidal effects through viral DNA deamination. Nonetheless, gNO administered by inhalation has not been demonstrated as safe or effective against acute viral infections or as a prophylactic viral treatment hitherto.

The present inventors have demonstrated that the use of supraphysiologic concentrations of gNO administered by inhalation may provide a broad spectrum, non-specific antiviral activity to be used at various stages of infection. The present inventors have tested two strains of human influenza (influenza A/victoria H3N2) and one strain of highly pathogenic avian influenza (H7N2), as well as human respiratory syncytial virus (rgRSV30), using the traditional plaque or fluorescence assays, and demonstrated that treating RSV and influenza with 160 ppm exogenous gaseous NO reduced their infectivity.

According to some embodiments of the present invention, a human in need of gNO inhalation is a human afflicted by a disease or disorder that is treatable by gNO. The range of treatable diseases and disorders spans ophthalmological, otolaryngological and/or an upper respiratory tract and/or a lower respiratory system diseases and disorders, as well as systemic medical conditions.

Exemplary diseases and disorders treatable by gNO include, without limitation, a heparin-protamine reaction, a traumatic injury, a traumatic injury to the respiratory tract, acidosis or sepsis, acute mountain sickness, acute pulmonary edema, acute pulmonary hypertension, acute pulmonary thromboembolism, adult respiratory distress syndrome, an acute pulmonary vasoconstriction, aspiration or inhalation injury or poisoning, asthma or status asthmaticus, bronchopulmonary dysplasia, hypoxia or chronic hypoxia, chronic pulmonary hypertension, chronic pulmonary thromboembolism, cystic fibrosis (CF), Aspergilosis, aspergilloma, Cryptococcosis, fat embolism of the lung, haline membrane disease, idiopathic or primary pulmonary hypertension, inflammation of the lung, perinatal aspiration syndrome, persistent pulmonary hypertension of a newborn and post cardiac surgery.

According to some embodiments of the present invention, exemplary treatable diseases or disorders include, without limitation, a bacterial-, viral- and/or fungal bronchiolitis, a bacterial-, viral- and/or fungal pharyngitis and/or laryngotracheitis, a bacterial-, viral- and/or fungal pneumonia, a bacterial-, viral- and/or fungal pulmonary infection, a bacterial-, viral- and/or fungal sinusitis, a bacterial-, viral- and/or fungal upper and/or lower respiratory tract infection, a bacterial-, viral- and/or fungal-exacerbated asthma, a respiratory syncytial viral infection, bronchiectasis, bronchitis, chronic obstructive lung disease (COPD), cystic fibrosis (CF), Aspergilosis, aspergilloma, Cryptococcosis, emphysema, otitis, a bacterial-, viral- and/or fungal otitis externa, otitis media, conjunctivitis, uveitis primary ciliary dyskinesia (PCD) and pulmonary aspergillosis (ABPA).

According to some embodiments of the present invention, the disease or disorder treatable by gNO is associated with a pathogenic microorganism. The pathogenic microorganisms, according to some embodiments of the present invention, can be, for example, Gram-negative bacteria, Gram-positive bacteria, viruses and viable virions, fungi and parasites. Exemplary pathogenic microorganisms include, but are not limited to, Acinetobacter baumarmii, Aspergillus niger, Bacteroides vufgatus, Burkhofderia cepacia, Candida albicans, Clostridium perfringes, Enteric Group 137, Enterococcus faecium, Enterohacter aerogenes, Escherichia cofi, Klebsiella pneumoniae, Klebsiella pneumoniae, Klebsiella pneumoniae, Mycobacteria tuberculosis, Pasteurella muftocida, Propbnibacterium acnes, Propbnibacterium granulosum, Proteus mirabilis, Providencia rusfigianii, Pseudomonas aeruginosa, Pseudomonas sp., Serratia marcesecens, Staphylococcus aureus, Staphylococcus aureus (FVL positive), Staphylococcus aureus (VNL positive), Staphylococcus aureus MRSA, Staphylococcus aureus MRSA, Staphylococcus aureus MRSA, Streptococci Group B, Streptococci Group D, Streptococci Group G, Streptococcipyrogenes rosenbach Group A, Streptococcus pneumoniae, Trichophyton meriagrophytes, Trichophyton rubrum, and Vibrio vuMucus.

Exemplary Gram-negative bacteria include, but are not limited to, Proteobacteria, Enterobacteriaceae, Acinetobacter baumannii, Bdellovibrio, Cyanobacteria, Enterobacter cloacae, Escherichia coli, Helicobacter, Helicobacter pylori, Hemophilus influenza, Klebsiella pneumonia, Legionella, Legionella pneumophila, Moraxella, Moraxella catarrhalis, Neisseria gonorrhoeae, Neisseria meningitides, Proteus mirabilis, Pseudomonas, Pseudomonas aeruginosa, Salmonella, Salmonella enteritidis, Salmonella typhi, Serratia marcescens, Shigella, Spirochaetes and Stenotrophomonas.

Exemplary Gram-positive bacteria include, but are not limited to, Bacillus species such as B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis and B. weihenstephanensis, Clostridium species such as C. acetobutylicum, C. aerotolerans, C. argentinense, C. baratii, C. beijerinckii, C. bifermentans, C. botulinum, C. butyricum, C. cadaveris, C. cellulolyticum, C. chauvoei, C. clostridioforme, C. colicanis, C. difficile, C. estertheticum, C. fallax, C. feseri, C. formicaceticum, C. histolyticum, C. innocuum, C. kluyveri, C. lavalense, C. ljungdahlii, C. novyi, C. oedematiens, C. paraputrificum, C. perfringens, C. phytofermentans, C. piliforme, C. ragsdalei, C. ramosum, C. scatologenes, C. septicum, C. sordellii, C. sporogenes, C. sticklandii, C. tertium, C. tetani, C. thermocellum, C. thermosaccharolyticum, C. tyrobutyricum, Corynebacterium species such as C. accolens, C. afermentans, C. amycolatum, C. aquaticum, C. argentoratense, C. auris, C. bovis, C. diphtherias, C. equi, C. flavescens, C. glucuronolyticum, C. glutamicum, C. granulosum, C. haemolyticum, C. halofytica, C. jeikeium, C. macginleyi, C. matruchotii, C. minutissimum, C. parvum, C. propinquum, C. pseudodiphtheriticum, C. pseudotuberculosis, C. pyogenes, C. renale, C. spec, C. striatum, C. tenuis, C. ulcerans, C. urealyticum, C. urealyticum and C. xerosis, Listeriai species such as L. grayi, L. innocua, L. ivanovii, L. monocytogenes, L. murrayi, L. seeligeri and L. welshimeri, Staphylococcus species such as S. arlettae, S. aureus, S. auricularis, S. capitis, S. caprae, S. carnosus, S. chromogenes, S. cohnii, S. condimenti, S. delphini, S. devriesei, S. epidermidis, S. equorum, S. felis, S. fleurettii, S. gallinarum, S. haemolyticus, S. hominis, S. hyicus, S. intermedius, S. kloosii, S. leei, S. lentus, S. lugdunensis, S. lutrae, S. massiliensis, S. microti, S. muscae, S. nepalensis, S. pasteuri, S. pettenkoferi, S. piscifermentans, S. pseudintermedius, S. pseudolugdunensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri and S. xylosus, and Streptococcus species such as S. agalactiae, S. anginosus, S. bovis, S. canis, S. constellatus, S. dysgalactiae, S. equinus, S. iniae, S. intermedius, S. mitis, S. mutans, S. oxalis, S. parasanguinis, S. peroris, S. pneumoniae, S. pyogenes, S. ratti, S. salivarius, S. sanguinis, S. sobrinus, S. suis, S. thermophilus, S. uberis, S. vestibularis, S. viridian and S. zooepidemicus.

As discussed hereinabove, and demonstrated in the Examples section that follows below, the disease or disorder which can be treated by effecting the method presented herein to a human subject, includes bacterial-, viral- and/or fungal bronchiolitis, bacterial-, viral- and/or fungal pharyngitis and/or laryngotracheitis, bacterial-, viral- and/or fungal sinusitis, bacterial-, viral- and/or fungal upper and/or lower respiratory tract infection, bacterial-, viral- and/or fungal-exacerbated asthma, bacterial-, viral-, fungal- and/or parasitic pneumonia, the common cold, cystic fibrosis related infections, aspergillosis, aspergilloma, respiratory syncytial viral infections, acidosis or sepsis, oral fungal infections, bronchitis, candidiasis of the oral cavity (thrush), canker sores, epiglottitis (supraglottitis), halitosis, herpes, laryngitis, laryngotracheitis, nasopharyngitis, otitis externa and otitis media, conjunctivitis, uveitis (and other eye infections) pharyngitis, pulmonary aspergillosis (ABPA), respiratory syncytial virus infections, rhinitis, rhinopharyingitis, rhinosinusitis, stomatitis, tonsillitis, tracheitis, tuberculosis, cryptococcosis and tympanitis.

According to some embodiments of the present invention, a human subject in need of gNO inhalation is a human subject in need of preemptive, preventative and prophylactic treatment of a disease or disorder as described herein. Hence, a subject not suffering from any current or manifested disease, and/or a subject that is suspected of being exposed to a pathogen, and/or a subject that suffers from one disease, is treated by the method(s) presented herein in order to prevent the occurrence of another disease or disorder.

As presented in the Examples section that follows below, the present inventors have contemplated treating bronchiolitis as this condition is defined hereinbelow. Hence, according to an aspect of some embodiments of the present invention, there is provided a method of treating a human subject suffering from bronchiolitis, which is effected by subjecting the subject to intermittent inhalation regimen, gNO at a concentration of at least 160 ppm, thereby treating bronchiolitis.

It is noted herein that the treatable bronchiolitis, according to some embodiments of the present invention, can be associated with a pathogenic microorganism or not associated therewith. It is therefore noted that the method presented herein can be used to treat idiopathic bronchiolitis, bacterial- and/or viral-induced bronchiolitis and/or bronchiolitis that is associated with other medical conditions such as, but not limited to, immune deficiency.

In some embodiments, the bronchiolitis is a viral-induced bronchiolitis. Exemplary viral infections that are known to be manifested by bronchiolitis include, but not limited to, respiratory syncytial viruses (RSV), rhinoviruses, coronaviruses, enteroviruses, influenza A and/or B viruses, parainfluenza 1, 2 and/or 3 viruses, bocaviruses, human metapneumoviruses, SARS and adenoviruses. However, infections caused by any other viruses are also contemplated.

According to an aspect of some embodiments of the present invention, there is provided a method of treating a human subject suffering from a disease or a disorder which is associated, directly or indirectly, with a pathogenic microorganism, as described herein. The method is effected by subjecting the subject to intermittent inhalation regimen of gNO as described herein.

According to another aspect of some embodiments of the present invention, there is provided a method of treating a human subject suffering from an ophthalmological, otolaryngological and/or upper respiratory tract disease or disorder, as described herein, which is effected by subjecting the subject to an inhalation regimen of gNO as described in any of the present embodiments.

According to some embodiments of the present invention, the otolaryngological and/or upper respiratory tract disease and disorder involves an infection or an inflammation of a bodily site selected from the group consisting of an ear cavity, a nasal cavity, a sinus cavity, an oral cavity, a pharynx, a epiglottis, a vocal cord, a trachea, an apex and an upper esophagus.

According to some embodiments of the present invention, the ophthalmological, otolaryngological and/or upper respiratory tract diseases and disorders include, without limitation, the common cold, a stomatognathic disease, amigdalitis, an oral fungal infection, bacterial-, viral- and/or fungal sinusitis, bronchitis, candidiasis of the oral cavity (thrush), canker sores, epiglottitis (supraglottitis), halitosis, herpes, laryngitis, laryngotracheitis, nasopharyngitis, otitis (externa and media), conjunctivitis, uveitis and other eye infections, pharyngitis, rhinitis, rhinopharyingitis, rhinosinusitis, stomatitis, tonsillitis, tracheitis, tracheitis and tympanitis.

According to another aspect of some embodiments of the present invention, there is provided a method of treating a human subject suffering from a disease or disorder of the lower respiratory system, as described herein, by an inhalation regimen of gNO as described in any of the embodiments herein.

According to some embodiments of the present invention, diseases and disorders of the lower respiratory system include, without limitation, an obstructive condition, a restrictive condition, a vascular disease and an infection, an inflammation due to inhalation of foreign matter and an inhaled particle poisoning.

According to some embodiments of the present invention, the obstructive condition includes, without limitation, a chronic obstructive lung disease (COPD), emphysema, bronchiolitis, bronchitis, asthma and viral, bacterial and fungal exacerbated asthma; the restrictive condition includes, without limitation, fibrosis, cystic fibrosis, sarcoidosis, alveolar damage and pleural effusion; the vascular disease includes, without limitation, pulmonary edema, pulmonary embolism and pulmonary hypertension; the infection includes, without limitation, respiratory syncytial virus infection, tuberculosis, a viral-, bacterial-, fungal-, and/or parasitic pneumonia, idiopathic pneumonia; and the inflammation due to inhalation of foreign matter and an inhaled particle poisoning includes, without limitation, smoke inhalation, asbestosis and exposure to particulate pollutants and fumes.

According to some embodiments of the present invention, any of the methods of treating or preventing a subject as described herein encompasses all of the conditions, disease and disorders described hereinabove for subjects in need of gNO inhalation.

It is noted herein that any of the methods described herein can be used beneficially to treat bronchiolitis, which occurs in infants and children. Administration by inhalation is considered to be a preferred method of for young patients and more so when invasive techniques are avoided.

Influenza of all sorts and types is also treatable by the methods presented herein, and where some embodiments being based on a relatively simple and noninvasive technique, these methods are particularly preferred in complicated and severe cases of influenza.

The methods presented herein are effective in treating asthma in children and adults, as well as treating COPD and CF.

The methods presented herein are fast and effective in treating a resent medical condition, disease or disorder. Moreover, the methods presented herein are effective in preventing the disease or disorder from taking hold in a subject which is prone to suffer from, contract or develop a disease or disorder which is associated with the respiratory tract. According to some embodiments, some methods of gNO inhalation are particularly useful in preventing a disease or disorder, while other methods are particularly effective in treating an existing disease or disorder.

Any of the methods presented herein can be used effectively to treat respiratory diseases or disorders that occur in humans which are diagnosed with medical conditions that adversely affect their innate immune system. Humans which are diagnosed with such medical conditions are said to be immuno-compromised or immuno-suppressed. It is noted herein that immuno-suppression may be a direct result of a pathogen, such as an HIV infection, or an indirect result such as immuno-suppression that occurs in cancer patients being treated with chemotherapeutic agents. Hence, according to some embodiments of the present invention, the methods presented herein are used to treat a present respiratory disease or disorder in immuno-compromised human subject.

Immuno-compromised or immuno-suppressed human subjects are intrinsically more susceptible to opportunistic infections, rendering them prone to suffer from respiratory diseases or disorders. Other incidents and conditions that render a human more susceptible to infections are associated with location, occupation, age, living and environmental conditions, close contact with large groups of people and livestock, close contact with sick people and the likes, all of which are encompassed in the context of the present invention as rendering a human subject prone to suffer from a respiratory disease or disorder.

According to some embodiments of the present invention, any of the methods presented herein are used to treat opportunistic infections in a human subject.

Exemplary opportunistic infections, which occur in human suffering from HIV, and can be treated or prevented by the methods presented herein include, without limitation Pneumocystis jiroveci infection, Pneumocystis carinii infection and pneumocystis pneumonia (a form of pneumonia caused by the yeast-like fungus).

Exemplary medical conditions which are associated with immunosuppression include AIDS, cancer, primary ciliary dyskinesia (PCD, also known as immotile ciliary syndrome or Kartagener Syndrome).

According to some embodiments of the present invention, any of the methods presented herein is used to treat a human subject suffering from AIDS.

According to some embodiments of the present invention, any of the methods presented herein are used to treat a human subject suffering from cancer.

According to some embodiments of the present invention, any of the methods presented herein can be used to treat or prevent an infection associated with immune deficiency. These include prevention/pre-emptive treatment and treatment of infections in oncology patients.

According to some embodiments of the present invention, in any of the methods presented herein the human subject is at risk of suffering from a nosocomial infection.

Exemplary groups of human subject which are prone to suffer respiratory disease or disorder due to general, environmental and occupational conditions include, without limitation, elderly people, medical staff and personnel (doctors, nurses, caretakers and the likes) of medical facilities and other care-giving homes and long-term facilities, commercial airline crew and personnel (pilots, flight attendants and the likes), livestock farmers and the likes.

According to some embodiments, the methods presented herein are used to treat or prevent nosocomial infections, such as infections stemming from direct-contact transmission, indirect-contact transmission, droplet transmission, airborne transmission, common vehicle transmission and vector borne transmission. Exemplary nosocomial infections are caused by antibiotic resistant bacteria such as carbapenem-resistant Klebsiella (KPC) or other Enterobacteriaceae, MRSA methicillin resistance Staph. Aureus, Group A Streptococcus, Staphylococcus aureus (methicillin sensitive or resistance), Neisseria meningitides of any serotype and the likes.

Hence, according to embodiments of the present invention, the methods presented herein can be used to prevent carriage, transmission and infection of pathogenic bacteria and antibiotic resistant pathogenic microorganisms.

According to some embodiments of the present invention, any of the methods of treatment presented herein further includes monitoring, during and following administration gNO, one or more of the parameters as described in any of the embodiments hereinabove.

According to some embodiments of the present invention, the disease or disorder is selected from the group consisting of a bacterial-, viral- and/or fungal bronchiolitis, a bacterial-, viral- and/or fungal pharyngitis and/or laryngotracheitis, a bacterial-, viral- and/or fungal pneumonia, a bacterial-, viral- and/or fungal sinusitis, a bacterial-, viral- and/or fungal upper and/or lower respiratory tract infection, a bacterial-, viral- and/or fungal-exacerbated asthma, a bacterial-, viral- and/or fungal conjunctivitis and uveitis, a respiratory syncytial viral infection, bronchiectasis, bronchitis, chronic obstructive lung disease (COPD), cystic fibrosis (CF), emphysema, otitis, otitis externa, otitis media, primary ciliary dyskinesia (PCD), aspergillosis, aspergilloma, pulmonary aspergillosis (ABPA) and cryptococcosis.

According to some embodiments of the present invention, the disease or disorder is an ophthalmological, otolaryngological and/or upper respiratory tract disease or disorder.

According to some embodiments of the present invention, the ophthalmological, otolaryngological and/or upper respiratory tract disease and disorder involves an infection or an inflammation of a bodily site selected from the group consisting of an ear cavity, a nasal cavity, an eye, a sinus cavity, an oral cavity, a pharynx, a epiglottis, a vocal cord, a trachea, an apex and an upper esophagus.

According to some embodiments of the present invention, the otolaryngological and/or upper respiratory tract disease and disorder is selected from the group consisting of a common cold, a stomatognathic disease, amigdalitis, an oral fungal infection, bacterial-, viral- and/or fungal sinusitis, bronchitis, candidiasis of the oral cavity (thrush), canker sores, epiglottitis (supraglottitis), halitosis, herpes, laryngitis, laryngotracheitis, nasopharyngitis, otitis, otitis externa, otitis media, conjunctivitis, uveitis, pharyngitis, rhinitis, rhinopharyingitis, rhinosinusitis, stomatitis, tonsillitis, tracheitis, tracheitis and tympanitis.

According to some embodiments of the present invention, the disease or disorder is a disease or disorder of the lower respiratory system of a human subject.

According to some embodiments of the present invention, the disease or disorder is selected from the group consisting of an obstructive condition, a restrictive condition, a vascular disease and an infection, an inflammation due to inhalation of foreign matter and an inhaled particle poisoning.

According to some embodiments of the present invention, the obstructive condition selected from the group consisting of a chronic obstructive lung disease (COPD), emphysema, bronchiolitis, bronchitis, asthma and viral, bacterial and fungal exacerbated asthma; the restrictive condition selected from the group consisting of fibrosis, cystic fibrosis, sarcoidosis, alveolar damage and pleural effusion; the vascular disease selected from the group consisting of pulmonary edema, pulmonary embolism and pulmonary hypertension; the infection selected from the group consisting of respiratory syncytial virus infection, tuberculosis, viral-, bacterial-, fungal-, and/or parasitic pneumonia, idiopathic pneumonia; and the inflammation due to inhalation of foreign matter and an inhaled particle poisoning selected from the group consisting of smoke inhalation, asbestosis and exposure to particulate pollutants and fumes.

According to some embodiments of the present invention, the disease or disorder is bronchiolitis.

According to some embodiments of the present invention, the bronchiolitis is associated with a virus.

According to some embodiments of the present invention, the virus is selected from the group consisting of a respiratory syncytial virus (RSV), a rhinovirus, a coronavirus, an enterovirus, an influenza A and/or B virus, a parainfluenza 1, 2 and/or 3 virus, a bocavirus, a human metapneumovirus, SARS and an adenovirus.

According to some embodiments of the present invention, the disease or disorder is asthma.

According to some embodiments of the present invention, the disease or disorder is cystic fibrosis.

According to some embodiments of the present invention, the disease or disorder is associated with an influenza virus.

According to some embodiments of the present invention, the disease or disorder is COPD.

According to some embodiments of the present invention, the disease or disorder selected from the group consisting of an acute respiratory disease or disorder, a chronic respiratory disease or disorder, an obstructive respiratory disease or disorder, an intrinsic or extrinsic restrictive respiratory disease or disorder, a pulmonary vascular disease or disorder, an infectious respiratory disease or disorder, an inflammatory respiratory disease or disorder, a pleural cavity disease or disorder, and a neonatal respiratory disease or disorder.

According to some embodiments of the present invention, the disease or disorder is associated with a pathogenic microorganism.

According to some embodiments of the present invention, the pathogenic microorganism is selected from the group consisting of a Gram-negative bacterium, a Gram-positive bacterium, a virus, a fungus and a parasite.

According to some embodiments of the present invention, the disease or disorder is selected from the group consisting of a bacterial-, viral- and/or fungal bronchiolitis, a bacterial-, viral- and/or fungal pharyngitis and/or laryngotracheitis, a bacterial-, viral- and/or fungal sinusitis, a bacterial-, viral- and/or fungal upper and/or lower respiratory tract infection, a bacterial-, viral- and/or fungal-exacerbated asthma, a bacterial-, viral-, fungal- and/or parasitic pneumonia, a common cold, a cystic fibrosis related infection, a respiratory syncytial viral infection, acidosis or sepsis, an oral fungal infection, aspergillosis, aspergilloma, cry ptococcosis, pulmonary aspergillosis (ABPA), cryptococcosis bronchitis, candidiasis of the oral cavity (thrush), canker sores, epiglottitis (supraglottitis), halitosis, herpes, laryngitis, laryngotracheitis, nasopharyngitis, otitis and otitis media, pharyngitis, respiratory syncytial virus infection, a bacterial-, viral- and/or fungal conjunctivitis and uveitis, rhinitis, rhinopharyingitis, rhinosinusitis, stomatitis, tonsillitis, tracheitis, tuberculosis and tympanitis.

According to some embodiments of the present invention, the method further comprises, or is effected while, monitoring, during and following the subjecting, at least one on-site parameter selected from the group consisting of:

a methemoglobin level (SpMet);

an oxygen saturation level (SpO₂);

an end tidal CO₂ level (ETCO₂); and

a fraction of inspired oxygen level (FiO₂),

and/or at least one off-site parameter selected from the group consisting of:

a serum nitrite level (NO₂⁻); and

an inflammatory cytokine plasma level,

in the subject, as these parameters are described herein.

According to some embodiments of the present invention, the method further comprises, or is effected while, monitoring, at least two of the parameters, as described herein.

According to some embodiments of the present invention, the method further comprises, or is effected while, monitoring all of the parameters.

According to some embodiments of the present invention, a change in the at least one of the parameters following the subjecting is less than 2 acceptable deviation units from a baseline, as described herein.

According to some embodiments of the present invention, a change in at least two of the parameters following the subjecting is less than 2 acceptable deviation units from a baseline.

According to some embodiments of the present invention, a change in all of the parameters following the subjecting is less than 2 acceptable deviation units from a baseline.

According to some embodiments of the present invention, a change in at least one of the on-site parameters following the subjecting is less than 2 acceptable deviation units from a baseline.

According to some embodiments of the present invention, a change in at least one of the off-site parameters following the subjecting is less than 2 acceptable deviation units from a baseline.

According to some of any of the embodiments of the present invention, the method further comprises, or is effected while, monitoring urine nitrite level in the subject, as described herein.

According to some embodiments of the present invention, the method further comprises, or is effected while, monitoring a change in the urine nitrite level following the subjecting is less than 2 acceptable deviation units from a baseline.

According to some of any of the embodiments of the present invention, the method further comprises, or is effected while, monitoring in the subject at least one off-site parameter selected from the group consisting of:

a hematological marker;

a vascular endothelial activation factor;

a coagulation parameter;

a serum creatinine level; and

a liver function marker, as these parameters are described herein, in the subject.

According to some embodiments of the present invention, a change in at least one of the off-site parameters following the subjecting is less than 2 acceptable deviation units from a baseline.

According to some of any of the embodiments of the present invention, the method further comprises, or is effected while, monitoring at least one off-site parameter selected from the group consisting of:

a hematological marker;

a vascular endothelial activation factor;

a coagulation parameter;

a serum creatinine level; and

a liver function marker, in the subject, as these parameters are described herein.

According to some embodiments of the present invention, a change in the at least one parameter following the subjecting is less than 2 acceptable deviation units from a baseline.

According to some of any of the embodiments of the present invention, the method further comprises, or is effected while, monitoring in the subject at least one on-site parameter selected from the group consisting of:

a vital sign; and

a pulmonary function, as these parameters are described herein.

According to some embodiments of the present invention, no deterioration is observed in the at least one parameter during and following the subjecting.

In some embodiments, the methods are effected while monitoring one, two, etc., or all of:

a methemoglobin level (SpMet) (an on-line parameter);

an end-tidal CO₂ level (ETCO₂) (an on-line parameter);

an oxygenation level or oxygen saturation level (SpO₂) (an on-line parameter);

an inflammatory cytokine plasma level (an off-line parameter); and

a serum nitrite/nitrate level (NO₂⁻/NO₃⁻) (an off-line parameter).

In some embodiments, no significant deviation from baseline, as described herein, is shown in at least one, two, three, four or all of the above parameters, when monitored, as described herein.

Other parameters and markers may be monitored as well, as presented hereinabove, while showing significant deviation from a baseline, and various general health indicators show no change to the worse, or an improvement, as presented hereinabove.

According to some embodiments of the present invention, in any of the methods of treatment presented herein, the gNO administration can be effected by an inhalation device which includes, without limitation, a stationary inhalation device, a portable inhaler, a metered-dose inhaler and an intubated inhaler.

An inhaler, according to some embodiments of the present invention, can generate spirometry data and adjust the treatment accordingly over time as provided, for example, in U.S. Pat. No. 5,724,986 and WO 2005/046426. The inhaler can modulate the subject's inhalation waveform to target specific lung sites. According to some embodiments of the present invention, a portable inhaler can deliver both rescue and maintenance doses of gNO at subject's selection or automatically according to a specified regimen.

According to some embodiments of the present invention, an exemplary inhalation device may include a delivery interface adaptable for inhalation by a human subject.

According to some embodiments of the present invention, the delivery interface includes a mask or a mouthpiece for delivery of the mixture of gases containing gNO to a respiratory organ of the subject.

According to some embodiments of the present invention, the inhalation device further includes a gNO analyzer positioned in proximity to the delivery interface for measuring the concentration of gNO, oxygen and nitrogen dioxide flowing to the delivery interface, wherein the analyzer is in communication with the controller.

According to some embodiments of the present invention, subjecting the subject to the method described herein is carried out by use of an inhalation device which can be any device which can deliver the mixture of gases containing gNO to a respiratory organ of the subject. An inhalation device, according to some embodiments of the present invention, includes, without limitation, a stationary inhalation device comprising tanks, gauges, tubing, a mask, controllers, values and the likes; a portable inhaler (inclusive of the aforementioned components), a metered-dose inhaler, a an atmospherically controlled enclosure, a respiration machine/system and an intubated inhalation/respiration machine/system. An atmospherically controlled enclosure includes, without limitation, a head enclosure (bubble), a full body enclosure or a room, wherein the atmosphere filling the enclosure can be controlled by flow, by a continuous or intermittent content exchange or any other form of controlling the gaseous mixture content thereof.

It is expected that during the life of a patent maturing from this application many relevant medical procedures involving inhalation of gNO will be developed and the scope of the term treatment by inhalation of gNO is intended to include all such new technologies a priori.

As used herein the term “about” refers to ±10%.

As used herein, the singular form “a”, “an” and “the” include plural references unless the context clearly dictates otherwise. For example, the term “a compound” or “at least one compound” may include a plurality of compounds, including mixtures thereof.

As used herein, the terms “patient” and “subject” are used interchangeably and generally refer to a human. In some embodiments, the patient has an infection. In some embodiments the patient does not have and infection. In some embodiments, the patient would benefit from improved lung function.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, and substantially ameliorating clinical or aesthetical symptoms of a condition.

As used herein, the term “preventing” includes substantially preventing the appearance of clinical or aesthetical symptoms of a condition, namely preemptive, preventative and prophylactic treatment.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

While this invention has been particularly shown and described with references to preferred embodiments thereof, it will be understood by those skilled in the art that various changes in form and details may be made therein without departing from the scope of the invention encompassed by the appended claims.

1. A method of delivering gaseous nitric oxide (gNO) to a patient, the method comprising (i) a first treatment period comprising administering at least 2000 ppm·hrs of gNO by inhalation over a period of about at least 5 days, wherein the first treatment period is followed by (ii) a second treatment period comprising administering at least 7,000 ppm·hrs of gNO by inhalation over a period of at least 3 months.

2. The method according to claim 1, wherein the patient has a respiratory disease.

3. The method according to claim 1, wherein at least 9,000 ppm·hrs of gNO by inhalation over a period of at least 3 months.

4. (canceled)

5. The method according to claim 1, wherein the second treatment period comprises administering at least 14,000 ppm·hrs of gNO by inhalation over a period of at least 6 months.

6. The method according to claim 3, comprising administering at least 16,000 ppm·hrs of gNO by inhalation over a period of at least 6 months.

7. The method according to claim 1, wherein during the first and/or second treatment periods the gNO is administered by intermittent inhalation, and wherein the intermittent inhalation comprises inhalation of the gNO for a first time period, followed by inhalation of no gNO for a second time period.

8. (canceled)

9. The method according to claim 7, wherein the first time period is from about 1 minute to about 60 minutes.

10. The method according to claim 9, wherein the first time period is about 30 minutes.

11. The method according to claim 7, wherein the second time period is from about 3 hours to about 5 hours.

12. The method according to claim 11, wherein the second time period is about 3.5 hours.

13. The method according to claim 1, wherein during the first treatment period the gNO is administered 1 to 6 times per day.

14. (canceled)

15. The method according to claim 7, wherein the intermittent inhalation during the first treatment period is performed 1 to 6 times per day.

16. (canceled)

17. The method according to claim 1, wherein the during the second treatment period gNO is administered 1 to 3 times per day.

18. (canceled)

19. (canceled)

20. The method according to claim 7, wherein the intermittent inhalation during the second treatment period is performed 1 to 3 times per day.

21. (canceled)

22. (canceled)

23. The method according to claim 1, wherein during the second treatment period the patient is administered gNO daily.

24. The method according to claim 1, wherein during the second treatment period the patient is administered gNO 1 to 5 times per week.

25. (canceled)

26. The method according to claim 1, wherein during the second treatment period the patient is administered gNO once per week.

27. The method according to claim 1, wherein during the second treatment period the patient is administered gNO every other week.

28. The method according to claim 1, wherein during the second treatment period the patient is administered gNO every other month.

29-39. (canceled)

40. The method according to claim 1, wherein during gNO administration NO₂ levels do not exceed 5 ppm, gNO concentration variations do not exceed 10%, and/or FiO₂/O₂ levels do not drop below 20%.

41-43. (canceled)

44. The method according to claim 40, wherein the fraction of inspired oxygen level (FiO₂) is greater than 21% but less than 100%.

45. (canceled)

46. The method according to claim 2, wherein the respiratory disease is associated with a pathogenic microorganism.

47. The method according to claim 46, wherein the pathogenic microorganism is selected from the group consisting of a Gram-negative bacterium, a Gram-positive bacterium, a virus, a fungus and a parasite.

48. The method according to claim 46, wherein the disease is selected from the group consisting of a bacterial bronchiolitis, viral bronchiolitis, fungal bronchiolitis, bacterial pharyngitis, viral pharyngitis, fungal pharyngitis, bacterial sinusitis, viral sinusitis, fungal sinusitis, a bacterial upper respiratory tract infection, viral upper respiratory tract infection, fungal upper respiratory tract infection, bacterial lower respiratory tract infection, viral lower respiratory tract infection, fungal lower respiratory tract infection, a bacterial-exacerbated asthma, viral-exacerbated asthma, fungal-exacerbated asthma, a bacterial pneumonia, viral pneumonia, fungal pneumonia, parasitic pneumonia, a common cold, a cystic fibrosis related infection, a respiratory syncytial viral infection, acidosis, sepsis, an oral fungal infection, aspergillosis, aspergilloma, cryptococcosis, pulmonary aspergillosis (ABPA), cryptococcosis bronchitis, candidiasis of the oral cavity (thrush), canker sores, epiglottitis (supraglottitis), halitosis, herpes, laryngitis, nasopharyngitis, otitis, otitis media, pharyngitis, rhinitis, rhinopharyingitis, rhinosinusitis, stomatitis, tonsillitis, tracheitis, tuberculosis and tympanitis.

49. The method according to claim 46, wherein the disease is non-tuberculous mycobacteria (NTM).

50. The method according to claim 2, wherein the disease is selected from the group consisting of chronic obstructive lung disease (COPD), cystic fibrosis (CF), emphysema, primary ciliary dyskinesia (PCD).

51. The method according to claim 50, wherein the disease is chronic obstructive lung disease (COPD).

52. The method according to claim 51, wherein the disease is cystic fibrosis (CF).

53-73. (canceled)

74. The method according to claim 47, wherein the virus is selected from the group consisting of a respiratory syncytial virus (RSV), a rhinovirus, a coronavirus, an enterovirus, an influenza A and/or B virus, a parainfluenza 1, 2 and/or 3 virus, a bocavirus, a human metapneumovirus, SARS and an adenovirus.

75. The method according to claim 74, wherein the virus is a coronavirus.
